(19) United States
(12) Patent Application Publication
(10) Pub. No.: US 2017/0065207 A1
(43) Pub. Date: Mar. 9, 2017

(54) ANTENNAS FOR SMALL IMDS

(71) Applicant: Cardiac Pacemakers, Inc., St. Paul, MN (US)

(72) Inventors: Daniel J. Landherr, Wyoming, MN (US); Niharika Varanasi, Blaine, MN (US); Keith R. Maile, New Brighton, MN (US); Jean M. Bobgan, Maple Grove, MN (US); Ron A. Balczewski, Bloomington, MN (US); William J. Linder, Golden Valley, MN (US)

(21) Appl. No.: 15/256,724

(22) Filed: Sep. 5, 2016

Related U.S. Application Data
(60) Provisional application No. 62/215,034, filed on Sep. 6, 2015.

(51) Int. Cl. A61B 5/07; A61N 1/372; A61B 5/00

(52) U.S. Cl. CPC A61B 5/076; A61B 5/0031; A61B 5/686; A61N 1/37229; A61B 2560/0468

(57) ABSTRACT

An implantable medical device (IMD) includes a core assembly having a housing with circuitry disposed therein. The IMD also includes an integrated electrode/antenna assembly. The integrated electrode/antenna assembly includes an electrode component and an antenna component.

### CROSS-REFERENCE TO RELATED APPLICATION

This application claims priority to Provisional Application No. 62/215,034, filed Sep. 6, 2015, which is herein incorporated by reference in its entirety.

### TECHNICAL FIELD

Aspects of embodiments of the present disclosure relate to implantable medical devices. More specifically, embodiments relate to antennas configured to enable implantable medical devices (“IMD”) to communicate with other devices.

### BACKGROUND

Implantable medical devices (IMDs) may be configured to sense physiological parameters and/or provide therapy and may include one or more electrodes for performing aspects of these functions. IMDs may also include antennas for communicating with other devices. Conventionally, devices such as programmers and wands have been used to communicate with IMDs, for example, to interrogate the IMDs, to cause the IMDs to take various actions (e.g., marking recordings of physiological parameters, initiating communications with other devices, etc.), and/or the like. Conventionally headers of IMDs may be small (e.g., on the order of 1/10- 1/30 of a wavelength that may be wished to be used for communication). Additionally, because higher frequencies such as, for example, frequencies greater than or equal to 1 GHz (e.g., Bluetooth frequencies) may be more readily absorbed by body tissue, antenna performance (e.g., gain) may be affected.

### SUMMARY

In an Example 1, an implantable medical device (IMD), comprises a core assembly comprising a housing having circuitry disposed therein; and an integrated electrode/antenna assembly.

In an Example 2, the IMD of Example 1, further comprising a header comprising an exterior surface that encloses an interior region; and a scaffold assembly configured to position at least a portion of the integrated electrode/antenna assembly within the interior region of the header.

In an Example 3, the IMD of any of Examples 1 and 2, wherein the integrated electrode/antenna assembly comprises an electrode component and an antenna component.

In an Example 4, the IMD of Example 3, wherein the electrode component comprises a conducting ribbon.

In an Example 5, the IMD of any of Examples 3 and 4, wherein the antenna component comprises a bent wire forming a bent monopole antenna.

In an Example 6, the IMD of any of Examples 3 and 4, wherein at least one of the antenna component and the electrode component comprises a conductive patch.

In an Example 7, the IMD of Example 6, wherein the conductive patch is electrically coupled, via a feedthrough, to the circuitry disposed within the core assembly.

In an Example 8, the IMD of any of Examples 6 and 7, further comprising an insulator that is disposed above at least a portion of the patch.

In an Example 9, a system comprises an implantable medical device (IMD) configured to be implanted within the body of a patient, the IMD comprising: a core assembly comprising a housing having circuitry disposed therein; a header comprising an exterior surface that encloses an interior region; an electrode disposed at least partially within the interior region of the header; and an antenna disposed at least partially within the interior region of the header; and a receiving device configured to communicate with the IMD via a wireless communication link.

In an Example 10, the system of Example 9, the wireless communication link comprising a Bluetooth link.

In an Example 11, the system of any of Examples 9 and 10, wherein the antenna is offset from the electrode.

In an Example 12, the system of Example 11, wherein the electrode is disposed adjacent a first outside surface of the header.

In an Example 13, the system of Example 12, wherein the IMD is configured to be implanted in a patient such that the first outside surface of the header faces in a direction toward the inside of the patient's body, and a second outside surface of the header faces in a direction away from the inside of the patient's body.

In an Example 14, the system of any of Examples 9-13, wherein the antenna comprises a conducting ribbon.

In an Example 15, the system of any of Examples 9-14, wherein the antenna comprises a bent wire forming a bent monopole antenna.

In an Example 16, an implantable medical device (IMD) comprises a core assembly comprising a housing having circuitry disposed therein; a header comprising an exterior surface that encloses an interior region; and an integrated electrode/antenna assembly, wherein the integrated electrode/antenna assembly comprises an electrode component and an antenna component.

In an Example 17, the IMD of Example 16, further comprising a scaffold assembly configured to position at least a portion of the integrated electrode/antenna assembly within the interior region of the header.

In an Example 18, the IMD of Example 17, wherein the antenna component is offset from the electrode component.

In an Example 19, the IMD of Example 18, wherein the electrode is disposed adjacent a first outside surface of the header.

In an Example 20, the IMD of Example 19, wherein the IMD is configured to be implanted in a patient such that the first outside surface of the header faces in a direction toward the inside of the patient's body, and a second outside surface of the header faces in a direction away from the inside of the patient's body.

In an Example 21, the IMD of Example 16, wherein the antenna component comprises a conducting ribbon.

In an Example 22, the IMD of Example 16, wherein the antenna component comprises a bent wire forming a bent monopole antenna.

In an Example 23, the IMD of Example 16, wherein the antenna component comprises: a driven element electrically coupled to the circuitry; and a reflector comprising a ground-plane disposed on at least a portion of an internal surface of the header, wherein the electrode component functions as a director.

In an Example 24, the IMD of Example 23, wherein the driven element comprises a biased field effect transistor (FET) or a folded dipole.

In an Example 25, the IMD of Example 16, wherein at least one of the antenna component and the electrode component comprises a conductive patch.

In an Example 26, the IMD of Example 25, wherein the conductive patch is electrically coupled, via a feedthrough, to the circuitry disposed within the core assembly.

In an Example 27, a system comprises an implantable medical device (IMD) configured to be implanted within the body of a patient, the IMD comprising: a core assembly comprising a housing having circuitry disposed therein; a header comprising an exterior surface that encloses an interior region; and an integrated electrode/antenna assembly; and a receiving device configured to communicate with the IMD via a wireless communication link.

In an Example 28, the system of Example 27, the wireless communication link comprising a Bluetooth link.

In an Example 29, the system of Example 27, wherein the integrated electrode/antenna assembly comprises an electrode component and an antenna component.

In an Example 30, the system of Example 29, wherein the electrode component comprises a conducting ribbon.

In an Example 31, the system of Example 29, wherein the antenna component comprises a bent wire forming a bent monopole antenna.

In an Example 32, the system of Example 29, wherein the antenna component comprises a conductive patch.

In an Example 33, an implantable medical device (IMD) comprises a core assembly comprising a housing having circuitry disposed therein; a header comprising an exterior surface that encloses an interior region; and an integrated electrode/antenna assembly at least partially disposed within the interior region of the header.

In an Example 34, the IMD of Example 33, wherein the integrated electrode/antenna assembly comprises an antenna component and an electrode component.

In an Example 35, the IMD of Example 34, wherein the antenna component comprises: a driven element electrically coupled to the circuitry; and a reflector comprising a ground-plane disposed on at least a portion of an internal surface of the header, wherein the electrode component functions as a director.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of a system having an implantable medical device (IMD) and a receiving device, in accordance with embodiments of the disclosure.

FIG. 2 is a perspective, partially-transparent, view illustration of an IMD, in accordance with embodiments of the disclosure.

FIG. 3A is a front-facing perspective view of a header and scaffold assembly, in accordance with embodiments of the disclosure.

FIG. 3B is a back-facing perspective view of the header and scaffold assembly shown in FIG. 3A, in accordance with embodiments of the disclosure.

FIG. 4A is a front schematic diagram of an illustrative antenna disposed in an IMD header, in accordance with embodiments of the disclosure.

FIG. 4B is a side schematic diagram of the illustrative antenna disposed in an IMD header, in accordance with embodiments of the disclosure.

FIG. 5 is a schematic diagram of an equivalent circuit of an antenna, in accordance with embodiments of the disclosure.

FIG. 6 is a schematic diagram of an illustrative antenna, in accordance with embodiments of the disclosure.

FIG. 7 is a schematic diagram depicting an illustrative IMD having a planar inverted-F antenna (PIFA), in accordance with embodiments of the disclosure.

FIG. 8 is a schematic diagram depicting an illustrative IMD with a patch antenna, in accordance with embodiments of the disclosure.

FIG. 9 is a schematic diagram depicting an illustrative IMD with a patch antenna, in accordance with embodiments of the disclosure.

FIG. 10 is a schematic diagram depicting an illustrative IMD with a slot antenna, in accordance with embodiments of the disclosure.

FIGS. 11A and 11B are schematic diagrams depicting illustrative antenna features, in accordance with embodiments of the disclosure.

FIGS. 12A, 12B, and 12C are, respectively, a top-view schematic diagram depicting an illustrative IMD, and side-view schematic diagrams depicting the illustrative IMD, having an integrated electrode/antenna assembly, in accordance with embodiments of the disclosure.

FIGS. 13-16 are schematic diagrams depicting IMDs having integrated electrode/antenna assemblies disposed within their headers, in accordance with embodiments of the disclosure.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosed subject matter as defined by the appended claims.

### DETAILED DESCRIPTION

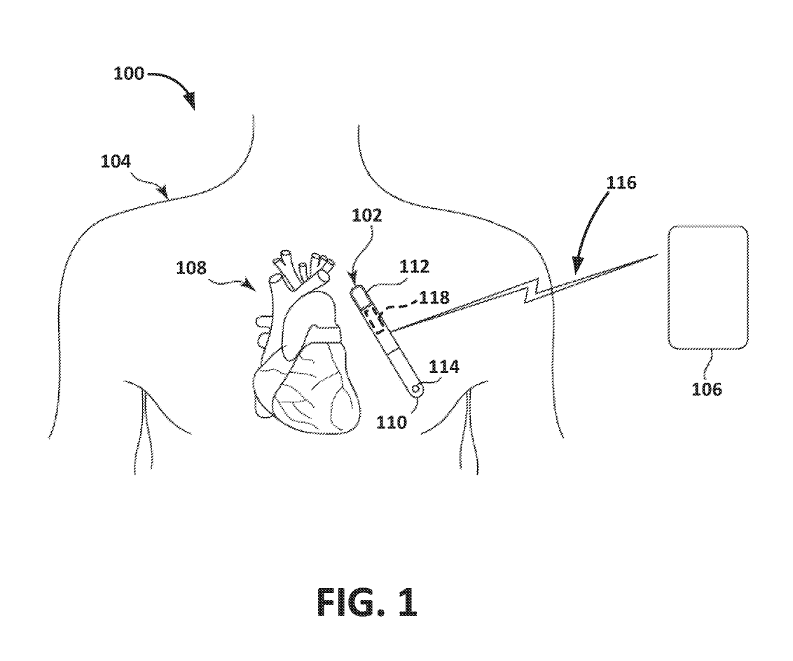

FIG. 1 is a schematic illustration of a system 100 including an implantable medical device (IMD) 102 implanted within a patient's body 104 and configured to communicate with a receiving device 106. The patient 104 may be a human, a dog, a pig, and/or any other animal having physiological parameters that can be recorded. In embodiments, the IMD 102 may be implanted subcutaneously within an implantation location or pocket in the patient's chest or abdomen and may be configured to monitor (e.g., sense and/or record) physiological parameters associated with the patient's heart 108. In embodiments, the IMD 102 may be an implantable cardiac monitor (ICM) (e.g., an implantable diagnostic monitor (IDM), an implantable loop recorder (ILR), etc.) configured to record physiological parameters such as, for example, one or more cardiac activation signals, heart sounds, blood pressure measurements, oxygen saturations, and/or the like. That is, for example, the IMD 102 may be configured to measure electrical signals of the patient's heart, which may be used to ascertain heart rate, heart rhythms, and/or the like.

In embodiments, the IMD 102 may be configured to monitor physiological parameters that may include one or more signals indicative of a patient's physical activity level and/or metabolic level, such as an acceleration signal. In embodiments, the IMD 102 may be configured to monitor physiological parameters associated with one or more other organs, systems, and/or the like. The IMD 102 may be configured to sense and/or record at regular intervals, continuously, and/or in response to a detected event. In embodiments, such a detected event may be detected by one or more sensors of the IMD 102, another IMD (not shown), an external device (e.g., the receiving device 106), and/or the like. In addition, the IMD 102 may be configured to detect a variety of physiological signals that may be used in connection with various diagnostic, therapeutic and/or monitoring implementations. For example, the IMD 102 may include sensors or circuitry for detecting respiratory system signals, cardiac system signals, heart sounds, and/or signals related to patient activity. In embodiments, the IMD 102 may be configured to sense intrathoracic impedance, from which various respiratory parameters may be derived, including, for example, respiratory tidal volume and minute ventilation. Sensors and associated circuitry may be incorporated in connection with the IMD 102 for detecting one or more body movement or body posture and/or position related signals. For example, accelerometers and/or GPS devices may be employed to detect patient activity, patient location, body orientation, and/or torso position.

For purposes of illustration, and not of limitation, various embodiments of devices that may be used to record physiological parameters in accordance with present disclosure are described herein in the context of IMDs that may be implanted under the skin in the chest region of a patient. However, it is contemplated that such devices may be implanted in any region of a patient, at any depth, to achieve any number of different objectives.

As shown, the IMD 102 may include a housing 110 having two electrodes 112 and 114 coupled thereto. According to embodiments, the IMD 102 may include any number of electrodes (and/or other types of sensors such as, e.g., thermometers, barometers, pressure sensors, optical sensors, motion sensors, and/or the like) in any number of various types of configurations, and the housing 110 may include any number of different shapes, sizes, and/or features. In embodiments, the IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory and communicate that recorded data to a receiving device 106. For example, in the case of an IDM, the IMD 102 may activate, record cardiac signals for a certain period of time, deactivate, and activate to communicate the recorded signals to the receiving device 106.

In various embodiments, the receiving device 106 may be, for example, a programmer, controller, patient monitoring system, and/or the like. Although illustrated, in FIG. 1, as an external device, the receiving device 106 may include an implantable device configured to communicate with the IMD 102 that may, for example, be a control device, another monitoring device, a pacemaker, an implantable defibrillator, a cardiac resynchronization therapy (CRT) device and/or the like, and may be an implantable medical device known in the art or later developed, for providing therapy and/or diagnostic data about the patient and/or the IMD 102. In various embodiments, the IMD 102 may be a pacemaker, an implantable cardioverter defibrillator (ICD) device, or a cardiac resynchronization therapy (CRT) device. In various embodiments, the IMD 102 may include both defibrillation and pacing/CRT capabilities (e.g., a CRT-D device).

The system 100 may be used to implement coordinated patient measuring and/or monitoring, diagnosis, and/or therapy in accordance with various embodiments. The system 100 may include, for example, one or more patient-internal medical devices, such as an IMD 102, and one or more patient-external medical devices, such as receiving device 106. In embodiments, the receiving device 106 may be configured to perform monitoring, and/or diagnosis and/or therapy functions external to the patient (i.e., not invasively implanted within the patient's body). The receiving device 106 may be positioned in the patient, on the patient, near the patient, or in any location external to the patient.

In embodiments, the IMD 102 and the receiving device 106 may communicate through a wireless link. For example, the IMD 102 and the receiving device 106 may be coupled through a short-range radio link 116, such as Bluetooth, IEEE 802.11, a proprietary wireless protocol, and/or the like. In embodiments, for example, the radio link 116 utilize Bluetooth Low Energy radio (Bluetooth 4.1), or a similar protocol, and may utilize an operating frequency in the range of 2.40 to 2.48 GHz. The communications link may facilitate uni-directional and/or bi-directional communication between the IMD 102 and the receiving device 106. Data and/or control signals may be transmitted between the IMD 102 and the receiving device 106 to coordinate the functions of the IMD 102 and/or the receiving device 106. In embodiments, patient data may be downloaded from one or more of the IMD 102 and the receiving device 106 periodically or on command. The physician and/or the patient may communicate with the IMD 102 and the receiving device 106, for example, to acquire patient data or to initiate, terminate or modify recording and/or therapy.

The communication link 116 may be facilitated, for example, by an antenna 118 disposed within, integrated with, and/or coupled to the IMD 102. The antenna 118 may include one or more antennas. The antenna 118 may be a bent monopole antenna, a patch antenna (e.g., a microstrip antenna, a planar inverted-F antenna (PIFA), etc.), a slot antenna, a planar inverted-F antenna, a combination of these, a modification of one or more of these, and/or the like. According to embodiments, the antenna 118 may be disposed, at least in part, within the IMD 102, integrated with a portion of the housing of the IMD 102, be, or include, at least a portion of the housing of the IMD 102, and/or the like.

The illustrative system 100 shown in FIG. 1 is not intended to suggest any limitation as to the scope of use or functionality of embodiments of the subject matter disclosed throughout this document. Neither should the illustrative system 100 be interpreted as having any dependency or requirement related to any single component or combination of components illustrated therein. For example, in embodiments, the illustrative system 100 may include additional components. Additionally, any one or more of the components depicted in FIG. 1 can be, in embodiments, integrated with various ones of the other components depicted therein (and/or components not illustrated). Any number of other components or combinations of components can be integrated with the illustrative system 100 depicted in FIG. 1, all of which are considered to be within the ambit of the disclosure.

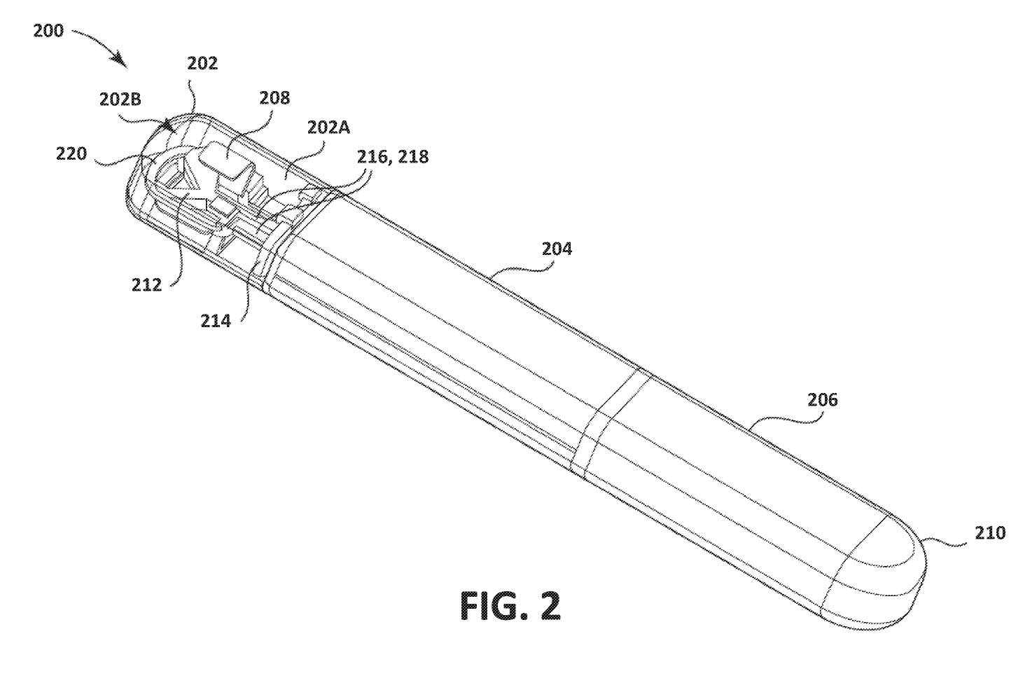

FIG. 2 is a schematic illustration of an implantable medical device (IMD) 200. As shown, the IMD 200 may include a header 202 arranged at or near a first end of a core assembly 204. The IMD 200 may include a battery assembly 206 arranged near a second end of the core assembly 204. The header 202 includes an exterior surface 202A that encloses an interior region 202B. The exterior surface 202A may contact a patient's bodily tissue when the IMD 200 is subcutaneously implanted in an implantation location such as, for example, in the patient's chest or abdomen. The interior region 202B of the header 202 may provide a space and house one or more circuit components positioned and supported by a scaffold assembly 212. As shown, the IMD 202 may include an electrode 208 disposed within the header 202, and an electrode 210 disposed at the opposite end of the IMD (e.g., coupled to an end of the battery assembly 206). According to embodiments, in order to enable sensing of physiological parameters within the patient, the electrode 208 may be positioned to be flush with the exterior surface 202A of the header 202. In other instances, the electrode 208 may be positioned by the scaffold assembly 212 to form a portion of the exterior surface 202A of the header 202.

As shown in FIG. 2, the scaffold assembly 212 positions and supports the electrode 208. Along with the electrode 208, the scaffold assembly 212 also may be configured to support and position one or more additional circuit components, such as an antenna 220. The scaffold assembly 212 may interface with a portion 214 of the core assembly 204, and may provide a throughput or alley for interconnects 216, 218. The interconnects 216, 218 may be configured to connect the circuitry components, such as the electrode 208 and antenna 220 positioned and supported by the scaffold assembly 212, to the integrated circuitry contained within the core assembly 204 of the IMD 200.

The illustrative IMD 200 shown in FIG. 2 is not intended to suggest any limitation as to the scope of use or functionality of embodiments of the subject matter disclosed throughout this document. Neither should the illustrative IMD 200 be interpreted as having any dependency or requirement related to any single component or combination of components illustrated therein. For example, in embodiments, the illustrative IMD 200 may include fewer components, additional components, and/or the like. Additionally, any one or more of the components depicted in FIG. 2 can be, in embodiments, integrated with various ones of the other components depicted therein (and/or components not illustrated). For example, in embodiments (as described below), the header 202 may not include an antenna, while an antenna may be disposed on, coupled to, and/or integrated with the core assembly 204. Any number of other components or combinations of components can be integrated with the illustrative IMD 200 depicted in FIG. 2, all of which are considered to be within the ambit of the disclosure.

### Monopole Antennas

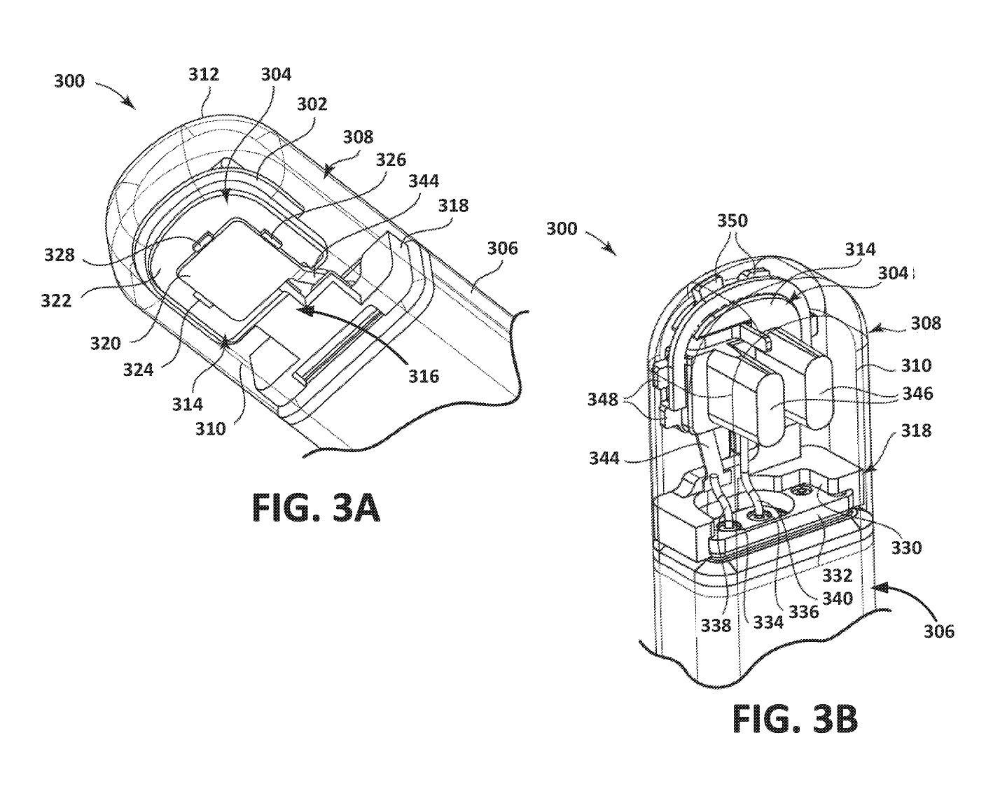

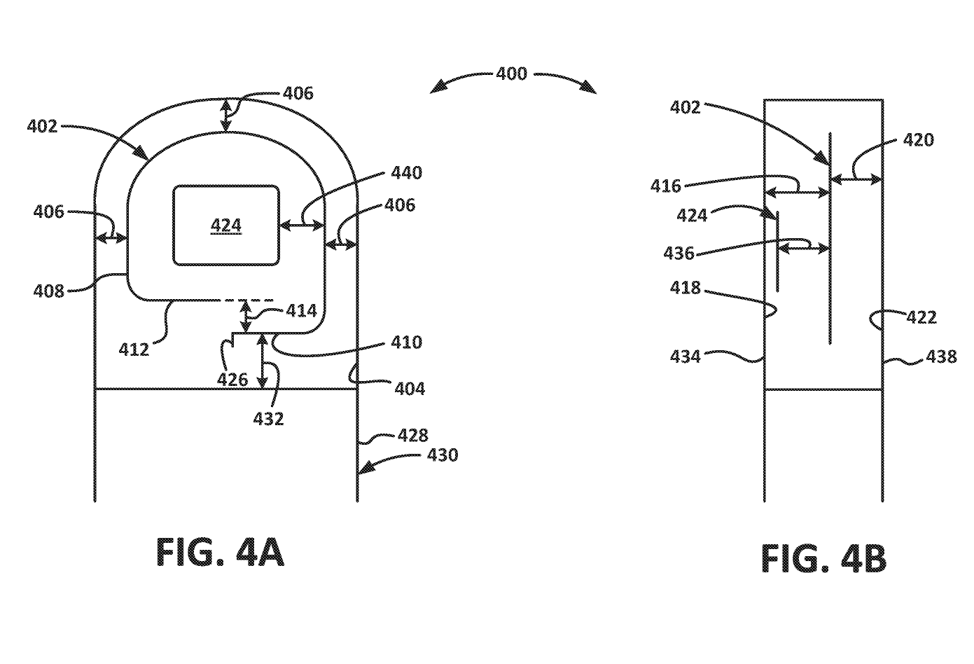

According to embodiments, an IMD (e.g., IMD 100 depicted in FIG. 1 and/or IMD 200 depicted in FIG. 2) may include a monopole antenna such as, for example, a shorted monopole antenna (e.g., a folded monopole antenna, a bent monopole, a planar inverted-F antenna (PIFA), etc.), as described, for example, in FIGS. 3A, 3B, 4A, and 4B. A monopole antenna may be configured to operate within a patient's body, outside of a patient's body, and/or between an interior and exterior of a patient's body. FIG. 3A is a partially-transparent, front-facing perspective view of a header 300 having a bent monopole antenna 302; and FIG. 3B is a partially-transparent, back-facing perspective view of the header 300 shown in FIG. 3A, in accordance with embodiments of the disclosure. In the embodiments illustrated in FIG. 3A, the bent monopole antenna is supported by a scaffold assembly 304, which is coupled to an end of a core assembly 306.

As shown in FIGS. 3A and 3B, the header 300 includes a housing 308 that encloses the internal portions of the header 300 from a patient's tissue when the header 300 is implanted as part of the IMD (e.g., as shown in FIG. 2 and as discussed above). The housing 308 includes an internal surface 310 and an opposite-facing external surface 312. The scaffold assembly 304 includes an upper portion 314, an intermediate portion 316, and a lower portion 318.

The upper portion 314 of the scaffold assembly 304 may be configure to support and position one or more circuit components. As shown in FIG. 3A, the upper portion 314 of the scaffold assembly 304 supports and positions the bent monopole antenna 302. In certain instances, positioning of the antenna 302 increase the functionality thereof by spatially arranging the broadcast direction(s) with respect to a receiving device configured to communicate with the antenna 302. As a result, and as shown in FIG. 3A, the antenna 302 may be at least partially circumferentially arranged around an exterior section of the upper portion 314 of the scaffold assembly 304. In embodiments, the positioning of the antenna 302 in this manner may allow for increasing the directional broadcast of the antenna while decreasing interference that may result from integrated circuitry, contained within the core assembly 306, which controls the antenna 302 broadcast.

Other arrangements of the antenna 302 may also be contemplated. For instance, the antenna 302 may be provided over a lesser or greater surface area of the upper portion 314 of the scaffold assembly 304. Further, the antenna 302 may be embedded in the upper portion 314 of the scaffold assembly 304 to allow for further protection of the antenna 302 by the scaffold assembly 304. The antenna 302 may be arranged along the sides of the intermediate portion 316 of the scaffold assembly 304. According to embodiments, the antenna 302 may be formed as a continuous or discontinuous structure.

Similar to the upper portion 314, the intermediate portion 316 of the scaffold assembly 304 may be configured to support and position a circuit component. As shown in FIG. 3A, the intermediate portion 316 of the scaffold assembly 304 supports and positions an electrode 320. In the illustrated embodiments, the electrode 320 is provided on a first surface 322 of the scaffold assembly 304. In embodiments, the electrode 320 may be secured in place on the scaffold assembly 304 by a push-in connection. The push-in connection may be accomplished by use of one or more push-in connectors 324, 326, 328 that secure the electrode 320. The push-in connectors 324, 326, 328 surround exterior portions of the electrode 320. In other embodiments, the scaffold assembly 304 may not include the push-in connectors 324, 326, 328. Instead, for example, the electrode 320 may include extensions, and the scaffold assembly 304 may include corresponding voids or gaps. In embodiments, a friction fit connection may be provided between these elements of the electrode 320 and the scaffold assembly 304 to secure the two together.

The scaffold assembly 304 may position and support the electrode 320 relative to the antenna 302. In certain instances, the antenna 302 may, at least in part, circumferentially surround the electrode 320, in the same plan as the electrode 320, a different plane from the electrode 320, partially in the same plane, and/or the like. In embodiments, the antenna 302 may be, or include, a wire (e.g., having a rounded cross-section), a ribbon (e.g., having a flat, rectangular cross section), and/or the like. In embodiments, the antenna 302 may be configured to lie in one or more planes, have any number of different widths and/or thicknesses, and/or the like. The antenna 302 may, for example, have a varying thickness and/or width, which may facilitate various desired resonance patterns.

As shown in FIG. 3B, the lower portion 318 of the scaffold assembly 304 may be configured to interface with the core assembly 306. The lower portion 318 may include a first mating feature 330 and the core assembly 306 may include a second mating feature 332. The first mating feature 330 and the second mating feature 332 may include one or more corresponding surfaces that engage to provide the interface between the scaffold assembly 304 and the core assembly 306. In certain instances, the first mating feature 330 and the second mating feature 332 may interface and provide a frictional fit to secure the scaffold assembly 304 together with the core assembly 306. In addition, an adhesive may be provided on one or more of the first mating feature 330 and the second mating feature 332 to secure the scaffold assembly 304 together with the core assembly 306.

The core assembly 306 may include one or more conduits 334, 336 that provide a feedthrough for at least one electrical connector or interconnect. As shown, in embodiments, two interconnects 338, 340 are provided and feed through the conduits 334, 336 along a second surface 342 (e.g., opposite the first surface 322) of the scaffold assembly 304. Each of the interconnects 338, 340 electrically connects a circuit component positioned and supported by the scaffold assembly 304 to the integrated circuitry contained within the core assembly 306. For example, one interconnect 338 electrically connects a tab portion 344 of the electrode 320 that pass from the front facing portion of the scaffold assembly 304 to the back facing portion of the scaffold assembly 304, and another interconnect 340 provides a connection between the antenna 302 and the integrated circuitry contained within the core assembly 306. The functionality of the antenna 302 may be controlled by integrated circuitry housed within the core assembly 306, and the antenna 302 may be electrically coupled to integrated circuitry contained within the core assembly 306 via the interconnect 340. Similarly, the functionality of the electrode 320 may be controlled by integrated circuitry housed within the core assembly 306, and the electrode 320 may be electrically coupled to the integrated circuitry via the interconnect 338.

As shown in FIG. 3B, embodiments of the scaffold assembly 304 may include one or more extensions 346 provided to contact the interior surface 310 of the housing 306. The extension 342 may include a single block structure, two separate (as shown), or three or more structures to support the scaffold assembly 302 against the second surface 310 of the housing 308. The extensions 346 may enhance the ability of the scaffold assembly 304 to position and support the electrode 320. In addition, the intermediate section 316 and/or the upper section 314 of the scaffold assembly 304 may include one or more side support members 348 and upper support members 350. The side support members 346 and the upper support members 350 may contact the internal surface 310 of the housing 308 to further ensure that the scaffold assembly 304 resists movement resulting from movement during manufacturing, normal bodily movement when implanted in a patient, and/or movement during the implantation procedure. The side support members 348 and the upper support members 350 may also further facilitate protection and/or positioning of the antenna 302. In embodiments, the scaffold assembly 304, antenna 302, and electrode 320 may be embedded in an epoxy, with may minimize tuning shift between environments (e.g., outside the patient's body and inside the patient's body).

The illustrative components shown in FIG. 3A and 3B are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIG. 3A and FIG. 3B (discussed in further detail below) may be, in embodiments, integrated with various ones of the other components depicted therein (and/or components not illustrated), all of which are considered to be within the ambit of the disclosed subject matter. For example, the electrode 320 may be secured to the scaffold assembly 304 using a clip. The scaffold assembly 304 may include further extensions to help support and contact portions of the housing 308. The scaffold assembly 304 may also, or alternatively, include addition circuit components (such as an additional electrode) and interconnects. In embodiments, each of the elements of the scaffold assembly 304 may be formed as a one-piece structure. Such a structure may be formed using an injection molding process, from pre-molded plastic, and/or formed of a like material and/or like process.

FIGS. 4A and 4B are a front and side schematic cross-sectional views, respectively, of an illustrative header 400 having a bent monopole antenna 402, in accordance with embodiments of the disclosure. As shown, the bent monopole antenna 402 may be configured to be wound, similar to a spiral, within the header 400. Although not illustrated in FIGS. 4A and 4B, the bent monopole antenna 402 may be supported by one or more support structures such as, for example, the scaffold assembly 304 depicted in FIGS. 3A and 3B. The scaffold assembly may be configured, for example, for holding an electrode to an inside surface during manufacturing and for ensuring consistent antenna location (and, thus, facilitating antenna tuning). In embodiments, the bent monopole antenna 402 may be configured to occupy a relatively large area such as, for example, by configuring the antenna 402 to be loosely wound and following an inside surface 404 of the header 400. A distance 406 between the antenna 402 and the inside surface 404 may be configured to be substantially constant (e.g., constant or within 5-10 mm of being constant) around the perimeter of the antenna 402, and/or for the length of a curved section 408 of the antenna 402.

Additionally, because different sections of the bent monopole antenna 402, set apart by discontinuities such as, for example, geometric discontinuities, material discontinuities, and/or the like, may resonate at different frequencies, a first straight section 410 may be configured to be a certain length, a second straight section 412 may be configured to be a certain length, and the curved section 408 may be configured to be a certain length. The bent monopole antenna 402 may be configured to have any number of coil turns, which may be, for example, fractional turns determined so as to cover certain amounts of a fractional circumference of a previous turn, to maintain a turn spacing 414, and/or the like. In embodiments, a fractional turn (less than a full turn) may be utilized. The antenna 402 may be configured to have a bend and/or turn as rounded as possible to minimize current crowding at angles/corners. For example, at least a portion of the antenna 402 may be configured to have a radius of curvature of approximately between 0.5 mm and 2 mm.

The bent monopole antenna 402 may be configured to lie in a single plane, as shown in FIG. 4B or in multiple planes. In embodiments, the bent monopole antenna 402 may be configured to maintain a constant or varying distance 416 between the antenna 402 and a first inside surface 418 and/or a constant or varying distance 420 between the antenna 402 and a second inside surface 422. In embodiments, the configuration of the antenna 402 may be designed to optimize a balance among a number of various parameters such as, for example, low power operations, surface area coverage, number of turns, resonance properties of various sections, and/or the like.

For example, the length of the entire antenna 402 may be defined by the ¼ wavelength at 2.4 GHz frequency in-vivo. The antenna 402 may be configured to be as far away as possible from an electrode 424 (e.g., the electrode 320 depicted in FIG. 3A) also disposed in the header 400, to minimize current cancellation. The antenna 402 may also be configured to be disposed far enough away from tissue (when the IMD is implanted) to minimize the detuning effect that the tissue can have on the antenna 402. This may be particularly true in the case that the tissue includes muscle, which has a higher conductivity, and thus may affect both the center frequency and cause increased losses. Accordingly, the antenna 402 may be configured to optimize the distance 406 from the antenna 402 to the inside surface 404 of the header 404 so that the distance 406 is as small as possible (resulting in as large of a turn as possible), while keeping the antenna 402 far enough away from the surrounding tissue to minimize capacitive coupling.

For example, in embodiments, the distance 406 may be between 0.2 mm and 2 mm. In other embodiments, the distance 406 may be between 0.5 mm and 1.5 mm. A mechanical offset may be introduced, which may define the distance 406. The mechanical offset may be, for example, between 50% and 90% (e.g., 75%, 80%, etc.). The offset may be defined, for example, by a ratio of conductivity between muscle (e.g., approximately 1.71), and fat (e.g., approximately 0.26) at 2.45 GHz. At the 85% point, for example, approximately the same amount of power is lost to fat as muscle. In that situation, for example, all tissue is still in the near field of the antenna 402, and the permittivity ratio of muscle (e.g., approximately 52.8) to fat (e.g., approximately 10.8) is approximately 80%. In general, the ratios of conductivity may be utilized in minimizing body losses and maximizing antenna efficiency. The permittivity of muscle may be utilized for shrinking the antenna length. Permittivity is a magnetic property and doesn't generally impact loss. However, using a ratio of permittivity that is equalized by the antenna position may make the antenna resonance consistent in more body types. In this manner, the ratio may facilitate enhancing both considerations—minimizing losses (conductivity) and providing a consistent resonant length (permittivity).

The antenna 402 may also be configured such that at least a portion of the antenna 402 is not coplanar with the electrode 424, as shown in FIG. 4B. For example, as shown in FIG. 4B, the electrode 424 may be disposed near the first inside surface 418. The electrode 424 may be positioned against the first inside surface 418, thereby being disposed adjacent a first outside surface 434 that is disposed opposite the first inside surface 418. In embodiments, the electrode 424 may be positioned flush with the inside surface 418, and/or may be configured to form a portion of the first outside surface 434. To maintain a relationship between the electrode 424 and the antenna 402 that is not coplanar, the antenna 402 may be offset from the first inside surface 418 in the direction of the second inside surface 422. The offset distance 436 between the electrode 424 and antenna 402 may be any number of different distances and may configured to enhance one or more of the performance characteristics described herein.

According to embodiments, the device may be implanted in the patient such that the first outside surface 434 is facing in a direction toward the inside of the patient's body (e.g., away from the surface of the patient's body nearest to the device), thereby increasing the ability of the electrode to detect electrical signals from within the patient's body. In this implantation position, the second outside surface 438 may be facing toward the outside of the patient's body (e.g., toward the surface of the patient's body that is nearest to the device) and, to the extent that the antenna 402 is offset so as to be disposed at or near the second inside surface 422, this implantation orientation may also enhance the ability of the antenna 402 to communicate with one or more devices external to the patient's body. In embodiments, the antenna 402 may be configured to be disposed adjacent the second outside surface 438 (e.g., by being disposed near or against the second inside surface 422), to form a portion of the second outside surface 438, and/or the like.

In embodiments, the antenna 402 may be at least partially coplanar with the electrode 424, but may be configured such that it is not parallel with an edge of the electrode 424, thereby minimizing inductive coupling. Additionally, the antenna 402 may be configured such that, when viewed from the front or back (e.g., a planar view), the electrode 424 does not overlap the antenna 402, so as to minimize capacitive coupling back to ground. In other words, the antenna 402 may be configured such that there is some gap 440 between the edge of the electrode 424 and the antenna 402, when viewed from a planar perspective, as shown, for example, in FIG. 4A.

The antenna 402 may be configured to maintain a certain spacing between the end of the antenna 402 and the ground connection 426 to the housing 428 (which may be referred to as, for example, “the can”) of the core assembly 430 so as to minimize the length of the antenna. It may also be desirable to minimize the current shunting of the first segment 410 to the core housing 428, particularly, for example, when the first segment 410 will be approximately parallel to a surface of the patient's body. Thus, for example, the antenna 402 may be configured to maximize a distance 432 between the end of the core housing 428 and the first segment 410. In embodiments, it may be desirable to maximize the length of the first segment 410, since the first segment 410 typically is the highest current region of the antenna 402.

According to embodiments, the overall size of the antenna 402 may be maximized as much as possible, in view of any one or more of the considerations discussed herein, as larger antennas tend to have better broadband characteristics. In this manner, for example, the antenna 402 may be optimized for use with a Bluetooth communication protocol, which spans 2.4 to 2.5 GHz. In embodiments, the antenna 402 may be constructed from a ribbon conductor to aid in manufacturability. A flat wire (e.g., ribbon) also has lower unit-length inductance, which may be a benefit for matching considerations. However, with ribbons, it may be desirable to consider surface conditions, as increased surface roughness may increase conductive loss due to skin-effect. Thus, for instance, it may be less desirable, in certain embodiments, to use a ribbon with an increased roughness to maximize surface area, since, at high frequencies, electrical current tends to flow on the surface of the ribbon. In embodiments, the orientation of a flat side of the conductor ribbon may be in the same plane as the plane of the antenna 402, in a plane orthogonal to the plane of the antenna 402, and/or the like. Additionally, the path of the antenna 402 may span more than one plane. In embodiments, the antenna 402 may twist axially.

In embodiments, high-dielectric materials, surrounded by body-contacting materials, may be used to increase electrical distance from the antenna 402 to muscle, thereby enabling the actual length of the antenna 402 to be shortened. In embodiments, for example, high-dielectric materials may facilitate shortening the length of the antenna because they typically have higher dielectric properties than epoxy and allow less coupling to the muscle because they move the muscle electrically farther away from the radiating element. Though most high-dielectric material generally will not have higher permittivity than muscle, many may have lower conductivity than that of muscle and a permittivity higher than that of epoxy.

According to embodiments, end-loading the antenna 402 may be used to add a lumped capacitance, which may be used for matching. Additionally, in embodiments, the antenna 402 may be configured such that the distal end of the antenna 402 is terminated at the core housing 428, thereby forming a magnetic, or loop, antenna 402 (e.g., for implementations that may have lower antenna impedance requirements). Additionally, as indicated above, the antenna 402 may be configured to be a multi-band antenna by tailoring certain segments (length, width, thickness, etc.), thereby changing the center frequencies of the segments. For example, in embodiments, a bent monopole antenna may include a primary loop (e.g., as described above in relation to FIGS. 3A, 3B, 4A, and 4B), and a number of additional turns of a wire that is thinner than the wire forming the primary loop. That is, for instance, the additional, thinner turns (e.g., 2 turns, 3 turns, 4 turns, 5 turns, and/or the like) may be made of wire that is approximately 1/10 the diameter of the wire forming the primary loop. In embodiments, an additional ground plane may be used as a reflector to minimize muscle losses. For example, a conducting sheet may be coupled to one of the internal surfaces 418 or 422 to serve as a ground plane.

The illustrative components shown in FIGS. 4A and 4B are not intended to suggest any limitation as to the scope of use or functionality of embodiments of the disclosed subject matter. Neither should the illustrative components be interpreted as having any dependency or requirement related to any single component or combination of components illustrated therein. For example, in embodiments, a variable-width ribbon may be used to construct the antenna 402, and/or a ribbon with a smooth or discontinuously smooth surface may be used to construct the antenna 402. Additionally, any one or more of the components depicted in FIG. 4A and 4B may be, in embodiments, integrated with various ones of the other components depicted therein (and/or components not illustrated), all of which are considered to be within the ambit of the disclosure. For example, the antenna 402 may be constructed by bending a long feedthrough wire.

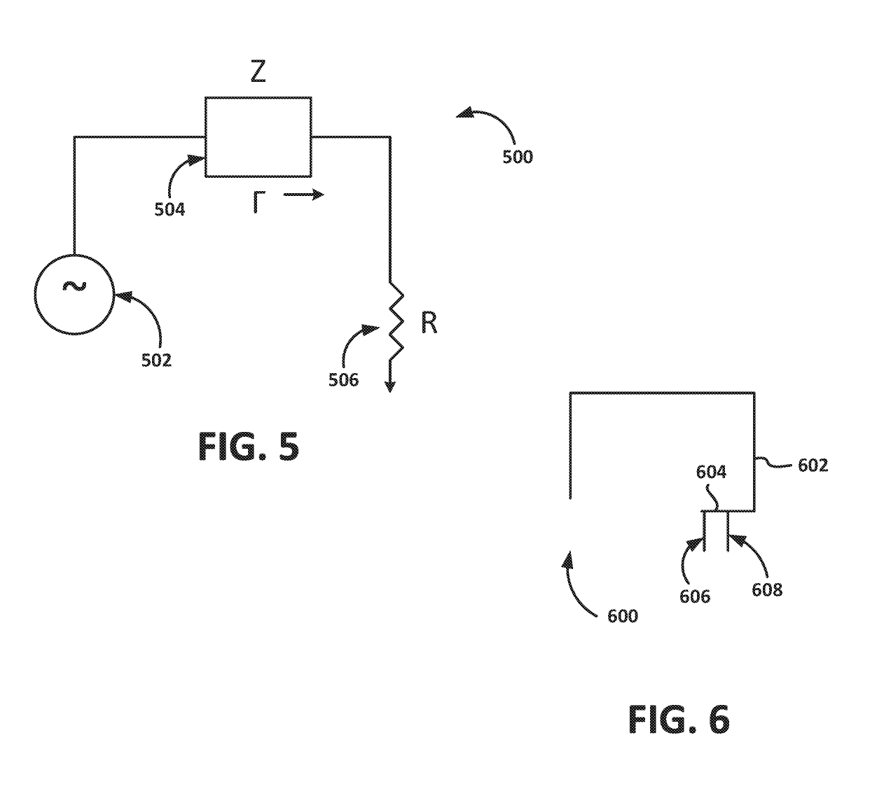

According to embodiments, an antenna for an IMD, as described herein, may be configured to have a particular characteristic impedance Z0, which may be, for example, between approximately 20 and 80 Ohms. For example, the antenna may be configured to minimize a reflection coefficient, Γ. In embodiments, the equivalent circuit 500, depicted in FIG. 5, may be used to model the antenna circuitry, wherein the power source 502 provides current to the antenna 504, which has an impedance Z. A resistor 506 may follow the antenna 504 in the current path. In embodiments, a reflection coefficient, Γ, may be modeled, and/or approximated, using a relation such as, for example:

$$Γ = \frac{( R - Z )}{( R + Z )} .$$

In this manner, the reflection coefficient, Γ, may be minimized by setting the impedance, Z, equal to the resistance, R (or approximately equal).

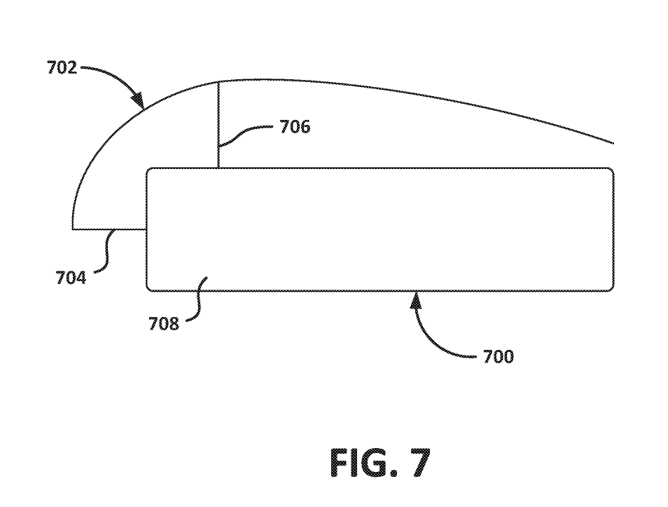

In embodiments, for example, this may be achieved using an F-type configuration for the antenna, as shown in FIG. 6. As shown, an antenna 600 includes a main loop section 602 that is connected to a first straight segment 604 that is coupled to a feedthrough wire 606. An exciting segment 608 is coupled to the first straight segment 604, as well, which results in the main loop section 602 acting as a resonant radiator, thereby minimizing an associated reflection coefficient, Γ. According to embodiments, any number of other configurations may be implemented for controlling characteristic impedances, reflective properties, and/or the like. For example, according to embodiments, another type of monopole antenna that may be utilized is a planar inverted-F antenna (PIFA), as indicated above. FIG. 7 is a schematic diagram depicting an illustrative IMD 700 having a PIFA 702, in accordance with embodiments of the disclosure. Current is provided to the PIFA 702 via a feedthrough wire 704, and the PIFA is grounded using a ground pin 706 coupled to at least a portion of the housing 708, which provides a grounding surface. According to embodiments, the PIFA may be formed using a wire, ribbon, sheet, and/or the like.

The illustrative components shown in FIGS. 6 and 7 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIGS. 6 and 7 may be, in embodiments, integrated with various ones of the other components depicted therein (and/or components not illustrated), all of which are considered to be within the ambit of the disclosure.

### Patch Antennas

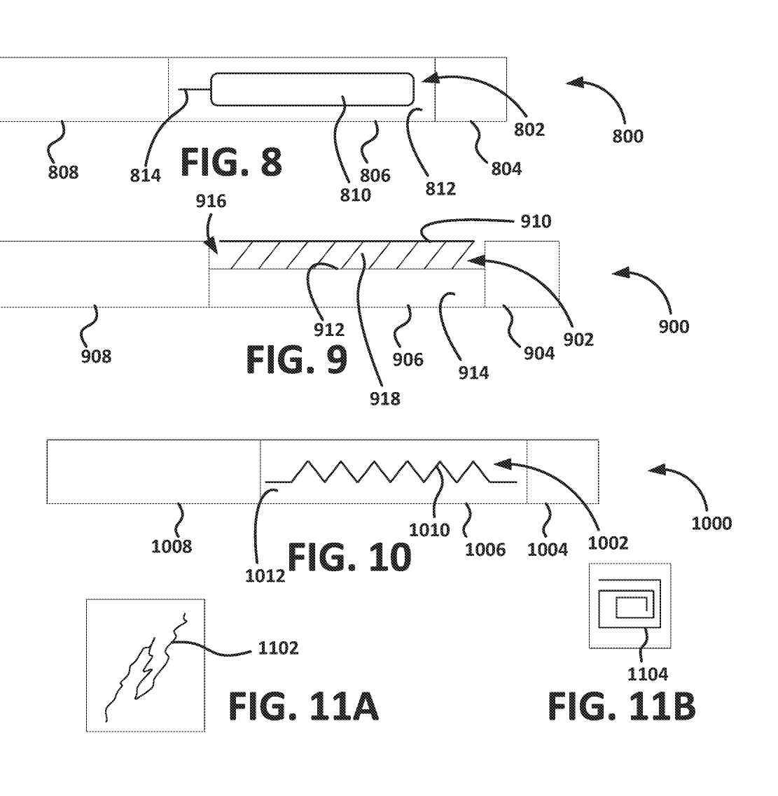

According to embodiments, an IMD (e.g., IMD 100 depicted in FIG. 1 and/or IMD 200 depicted in FIG. 2) may include a patch antenna, as depicted, for example, in FIGS. 8 and 9. FIG. 8 is a schematic block diagram depicting an illustrative IMD 800 with a patch antenna 802, in accordance with embodiments of the disclosure. The illustrative IMD 800 includes a header 804, a core assembly 806, and a battery assembly 808. The patch antenna 802 includes a patch 810 disposed above a ground plane 812, and may be driven using a feedthrough wire 814 that feeds through to circuitry disposed within the core assembly 806. The patch 810 radiates against the ground plane 812, and may include a microstrip, a conducting sheet (e.g., metal), and/or the like. The ground plane 812 may be the housing of the core assembly 806, a conductive layer disposed on the housing, and/or the like. In embodiments, a dielectric layer may be disposed between the housing and the patch 810.

According to embodiments, the patch may be recessed into an outer housing of the IMD such as, for example, is depicted in FIG. 9 is a schematic block diagram depicting an illustrative IMD 900 with a patch antenna 902, in accordance with embodiments of the disclosure. The illustrative IMD 900 includes a header 904, a core assembly 906, and a battery assembly 908. The patch antenna 902 includes a patch 910 disposed above a ground plane 912. The ground plane 912 may be at least a portion of the housing 914 of the core assembly, which may include a recessed region (e.g., a depression, notch, etc.) 916. In embodiments, the housing 914 may be configured such that the recessed region 916 enables the patch 910 to be at least substantially flush with the remainder of the housing of the IMD 900. As shown, a dielectric layer 918 may be disposed between the patch 910 and the housing 914.

In embodiments, feedthroughs may be configured to facilitate connecting a patch antenna interconnect to the appropriate circuitry within the core assembly of the IMD. In other embodiments, the antenna interconnect may be connected to the circuitry via the header, thereby utilizing traditional feedthroughs from the header to the core assembly. In embodiments, the antenna interconnect may be part of the antenna.

The illustrative components shown in FIGS. 8 and 9 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIGS. 8 and 9 may be, in embodiments, integrated with various ones of the other components depicted therein (and/or components not illustrated), all of which are considered to be within the ambit of the disclosure.

### Slot Antennas

According to embodiments, an IMD (e.g., IMD 100 depicted in FIG. 1 and/or IMD 200 depicted in FIG. 2) may include a slot antenna, as depicted, for example, in FIGS. 10-11B. FIG. 10 is a schematic block diagram depicting an illustrative IMD 1000 with a slot antenna 1002, in accordance with embodiments of the disclosure. The illustrative IMD 1000 includes a header 1004, a core assembly 1006, and a battery assembly 1008. The slot antenna 1002 includes a slot 1010 defined in a conductive surface 1012. In embodiments, the conductive surface 1012, may be recessed into the housing of the core assembly 1006. The surface 1012 may be a well-defined ground plane separated from the housing of the core assembly 1006 by a dielectric layer. In embodiments, the slot 1010 may be configured according to any number of different shapes. For example, the slot 1010 may be configured according to a zigzag shape, as shown in FIG. 10. As will be understood by those having skill in relevant arts, in a slot antenna, the slot itself, which may be cut into a ground plane, radiates the electromagnetic waves.

According to embodiments, any number of different types of features and/or modifications may be employed to facilitate minimizing undesirable effects resulting from interactions between electrodes and antennas and/or enhancing desirable effects resulting from interactions between electrodes and antennas. For example, as shown in FIG. 11A, an antenna feature 1102 (e.g., a patch of a patch antenna, a slot of a slot antenna, etc.) may be configured according to irregular path shapes, so as, for example, to minimize eddy currents. As shown in FIG. 11B, such an antenna feature 1104 may be configured according to a spiral pattern. In embodiments, an antenna may be disposed behind an electrode, in the same shape as the slot (e.g., slot 1102, 1104, etc.). In embodiments, electrodes may be placed at the nodes (e.g., quiet regions) of the antenna. Lumped components may be used to increase various properties. That is, for example, a lumped top-hat component may be used to increase capacitance. In another example, a ferrite bead disposed on a base wire may be used to improve inductance. In embodiments, a slotted or ribbon type of electrode may be utilized to provide better separation from the antenna.

The illustrative components shown in FIGS. 10, 11A, and 11B are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIGS. 10, 11A, and 11B may be, in embodiments, integrated with various ones of the other components depicted therein (and/or components not illustrated), all of which are considered to be within the ambit of the disclosure.

### Integrated Electrode/Antenna Assemblies

According to embodiments, an IMD, as described herein, may utilize an integrated electrode/antenna assembly for facilitating communication. Such a solution may, for example, be implemented to facilitate Bluetooth communications between an IMD (e.g., the IMD 102 depicted in FIG. 1) and a receiving device (e.g., the receiving device 106 depicted in FIG. 1). According to embodiments, an integrated electrode/antenna assembly may include an antenna component (e.g., a monopole antenna, a patch antenna, and/or the like), and an electrode component (e.g., a sensing electrode, a stimulation electrode, and/or the like).

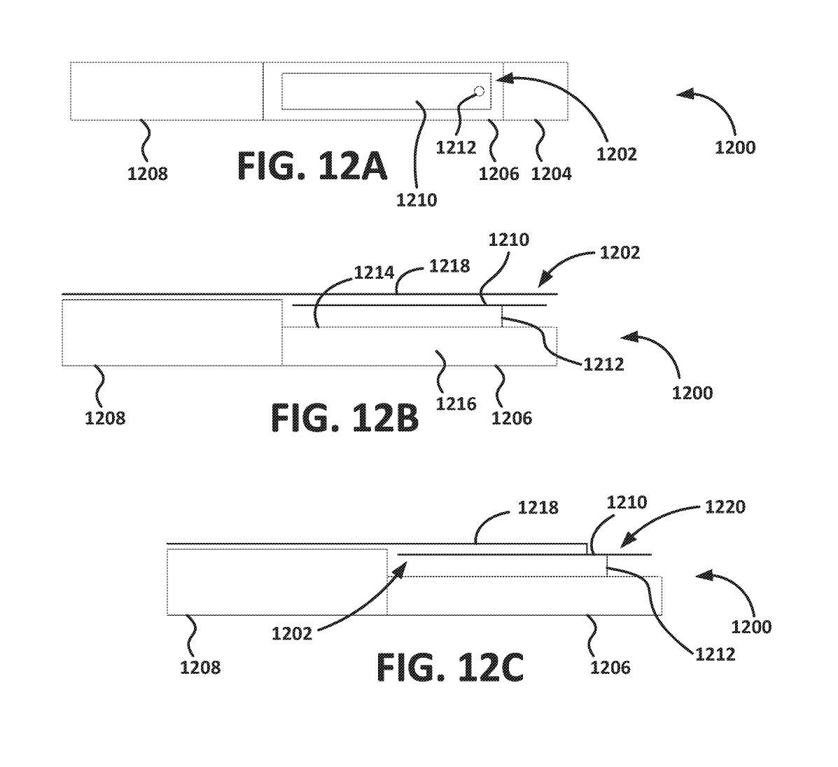

FIGS. 12A, 12B, and 12C are, respectively, a top-view block schematic diagram depicting an illustrative IMD 1200, and side-view block schematic diagrams depicting the illustrative IMD 1200, having an integrated electrode/antenna assembly 1202, in accordance with embodiments of the disclosure. As shown, the illustrative IMD 1200 includes a header 1204, a core assembly 1206, and a battery assembly 1208. In embodiments, the illustrated components 1204, 1206, and 1208, may instead include a header 1204, 1206, and a core assembly 1208, or any other combination and/or arrangement of components. The integrated electrode/antenna assembly 1202 includes a patch 1210 electrically coupled, via a feedthrough pin 1212, to the core assembly 1206. The patch 1210 may be configured to radiate against a ground plane 1214, which may be disposed on (and/or include at least a portion of) the housing 1216 of the IMD 1200. In embodiments, a dielectric layer (not shown) may be disposed between the housing 1216 and the ground plane 1214. Additionally, the patch 1210 may be configured to function as a sense and/or therapy electrode, such as, by being configured to sense electrical signals (e.g., electrocardiograms, respiratory signals, etc.), and/or deliver therapy (e.g., stimulation therapy).

As shown in FIG. 12B, an insulator 1218 may be disposed over the patch 1210 to insulate the patch 1210, protect the patch, and/or the like. In embodiments, the insulator 1218 may be configured to enable detection of signals by the patch 1210, delivery of therapy by the patch 1210, and/or other functions that would be performed by an electrode. For example, the insulator 1218 may include one or more regions that are thinner or made of a conducting, or partially-conducting, material. In embodiments, as shown in FIG. 12C, the insulator 1218 may be configured so that it doesn't cover the entire patch 1210—that is, for example, the patch 1210 may include an exposed region 1220 that may be brought into contact with the tissue of the patient so as to enhance the functionality of the patch 1210 as a sensing/therapy electrode.

The illustrative components shown in FIGS. 12A, 12B, and 12C are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIGS. 12A, 12B, and 12C may be, in embodiments, integrated with various ones of the other components depicted therein (and/or components not illustrated), all of which are considered to be within the ambit of the disclosure.

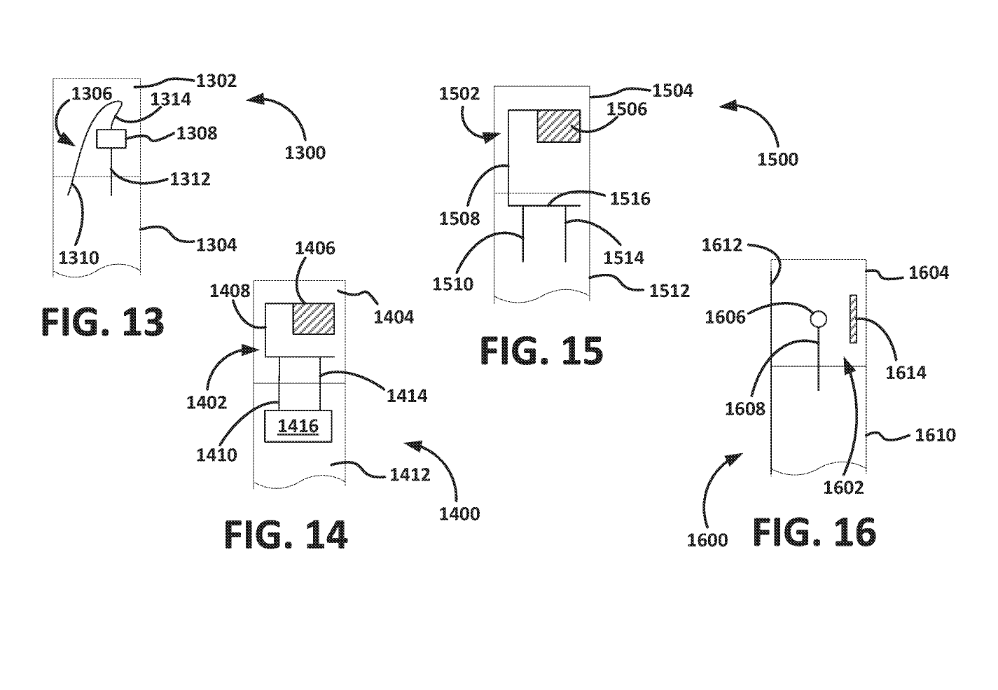

According to embodiments, an integrated electrode/antenna assembly may be disposed in a header of an IMD, such as is depicted, for example, in FIGS. 13-16. FIGS. 13-16 are block schematic diagrams depicting IMDs having integrated electrode/antenna assemblies disposed within their headers, in accordance with embodiments of the disclosure. As described above, in embodiments, the electrode may be, include, or be included in, the same component as the antenna. The electrode/antenna assemblies described with regard to FIGS. 13-16 may include an antenna component and an electrode component and, in embodiments, the antenna component and/or the electrode component may be supported by a scaffold assembly, which may be, include, or be included in, the scaffold assembly 212 depicted in FIGS. 2-3B. In this manner, for example, in embodiments depicted in FIGS. 13-16, the antenna component and the electrode component may be supported by a scaffold assembly so as to have respective orientations identical to, or similar to, those described herein with regards to FIGS. 3-4B.

As shown, in FIG. 13, the depicted IMD 1300 includes a header 1302 disposed on an end of a core assembly 1304. The integrated electrode/antenna assembly 1306 includes an electrode component 1308 disposed in the header 1302, an antenna connection wire 1310 coupled to the electrode component 1308 and configured to function as an antenna and serve as an antenna feedthrough to the core assembly 1304; and an electrode connection 1312 coupled to the electrode component 1308 and configured to function as an electrode feedthrough to the core assembly 1304. As shown in FIG. 13, the antenna connection 1310 may include a loop portion 1314 that may be configured to function in a similar manner as a bent monopole antenna. In embodiments, the interaction between the antenna connection 1310 and the electrode component 1308 may be configured to function essentially as a patch/monopole hybrid. A ground plane may be disposed on one side of the integrated electrode/antenna assembly 1306 to function as a reflector. The ground plane may be, or be coupled to, an internal surface of the header 1302 such as, for example is explained with respect to FIG. 4B. In embodiments, the integrated electrode/antenna assembly 1306 may simply include a single length of wire that terminates, at both ends, in the core assembly 1304 and has a loop feature within the header 1302.

FIG. 14 is a block schematic diagram depicting another IMD 1400 having an integrated electrode/antenna assembly 1402 disposed within its header 1404, in accordance with embodiments of the disclosure. As shown, the integrated electrode/antenna assembly 1402 includes an electrode component 1406 disposed in the header; an antenna component 1408 disposed in the header 1404 and coupled, at one end, to the electrode component 1406; an antenna connection wire 1410 coupled to the antenna component 1408 and configured to function as an antenna feedthrough to the core assembly 1412; and an electrode connection wire 1414 coupled to the electrode component 1406 via the antenna component 1408, and configured to function as an electrode feedthrough to the core assembly 1412. As shown in FIG. 14, a filter 1416 may be disposed between the antenna connection wire 1410 and the electrode connection wire 1414. The filter 1416 may be configured to filter interfering electrode signals (e.g., sensed electrical signals) from communication signals generated and/or received by the antenna component 1408, and/or to filter interfering communication signals (e.g., transmitted/received signals) from sense/therapy signals generated and/or received by the electrode component 1406, and/or the like.

FIG. 15 is a block schematic diagram depicting another IMD 1500 having an integrated electrode/antenna assembly 1502 disposed at least partially within its header 1504, in accordance with embodiments of the disclosure. As shown, the integrated electrode/antenna assembly 1502 includes an electrode component 1506 disposed in the header 1504; an antenna component 1508 partially disposed in the header 1504 and coupled, at one end, to the electrode component 1506; an antenna connection wire 1510 coupled to the antenna component 1508 and configured to function as an antenna feedthrough to the core assembly 1512; and an electrode connection wire 1514 coupled to the electrode component 1506 via the antenna component 1508, and configured to function as an electrode feedthrough to the core assembly 1512. As shown in FIG. 15, a “bias-T” section 1516 of the antenna portion 1508 may be disposed within the core assembly 1512 and may be configured to function similarly to a bias-T assembly, by duplexing the antenna signal and the electrode signal. That is, for example, the “bias-T” section 1516 may facilitate moving communication signals to and from the antenna portion 1508 and sensing and/or therapy signals to and from the electrode component 1506 along the same wire 1516.

FIG. 16 is a block schematic diagram depicting another IMD 1600 having an integrated electrode/antenna assembly 1602 disposed at least partially within its header 1604, in accordance with embodiments of the disclosure. As shown, the integrated electrode/antenna assembly 1602 may be similar, for example, to a Yagi-Uda antenna, and includes a driven element 1606 that may be coupled, via an antenna connector 1608 to a transmitter/receiver, transceiver, and/or the like, in the core assembly 1610. The driven element 1606 may include, for example, a folded dipole, a biased field effect transistor (FET), and/or the like. The integrated electrode/antenna assembly 1602 also includes a reflector 1612, which may be a ground-plane such as, for example, a conductive sheet disposed on an internal surface of the header 1604. In embodiments, the reflector 1612 may be an extension of the housing of the core assembly 1610 itself, and may be, for example, laminated into a ceramic and/or epoxy structure. An electrode component 1614 may be configured to function as a director, and may be disposed in the header 1604 in such a way as to perform that function, as well as functions as a sensing/therapy electrode.

The illustrative components shown in FIGS. 15 and 16 are not intended to suggest any limitation as to the scope of use or functionality of embodiments of the disclosed subject matter. Neither should the illustrative components be interpreted as having any dependency or requirement related to any single component or combination of components illustrated therein. Additionally, any one or more of the components depicted in FIGS. 15 and 16 may be, in embodiments, integrated with various ones of the other components depicted therein (and/or components not illustrated), all of which are considered to be within the ambit of the disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. In embodiments, for example, the antennal may be a single or multi-turn loop antenna that may, for example, terminate at the header, a surface of the device, go through a feedthrough defined in the surface of the device, and/or the like. Accordingly, the scope of the present disclosure is intended to embrace all such alternatives, modifications, and variations as fall within the scope of the claims, together with all equivalents thereof.

We claim:

1. An implantable medical device (IMD), comprising:
a core assembly comprising a housing having circuitry disposed therein;
a header comprising an exterior surface that encloses an interior region; and
an integrated electrode/antenna assembly, wherein the integrated electrode/antenna assembly comprises an electrode component and an antenna component.

2. The IMD of claim 1, further comprising a scaffold assembly configured to position at least a portion of the integrated electrode/antenna assembly within the interior region of the header.

3. The IMD of claim 2, wherein the antenna component is offset from the electrode component.

4. The IMD of claim 3, wherein the electrode is disposed adjacent a first outside surface of the header.

5. The IMD of claim 4, wherein the IMD is configured to be implanted in a patient such that the first outside surface of the header faces in a direction toward the inside of the patient's body, and a second outside surface of the header faces in a direction away from the inside of the patient's body.

6. The IMD of claim 1, wherein the antenna component comprises a conducting ribbon.

7. The IMD of claim 1, wherein the antenna component comprises a bent wire forming a bent monopole antenna.

8. The IMD of claim 1, wherein the antenna component comprises:
a driven element electrically coupled to the circuitry; and
a reflector comprising a ground-plane disposed on at least a portion of an internal surface of the header, wherein the electrode component functions as a director.

9. The IMD of claim 8, wherein the driven element comprises a biased field effect transistor (FET) or a folded dipole.

10. The IMD of claim 1, wherein at least one of the antenna component and the electrode component comprises a conductive patch.

11. The IMD of claim 10, wherein the conductive patch is electrically coupled, via a feedthrough, to the circuitry disposed within the core assembly.

12. A system, comprising:
an implantable medical device (IMD) configured to be implanted within the body of a patient, the IMD comprising:
a core assembly comprising a housing having circuitry disposed therein;
a header comprising an exterior surface that encloses an interior region; and
an integrated electrode/antenna assembly; and
a receiving device configured to communicate with the IMD via a wireless communication link.

13. The system of claim 12, the wireless communication link comprising a Bluetooth link.

14. The system of claim 12, wherein the integrated electrode/antenna assembly comprises an electrode component and an antenna component.

15. The system of claim 14, wherein the electrode component comprises a conducting ribbon.

16. The system of claim 14, wherein the antenna component comprises a bent wire forming a bent monopole antenna.

17. The system of claim 14, wherein the antenna component comprises a conductive patch.

18. An implantable medical device (IMD), comprising:
a core assembly comprising a housing having circuitry disposed therein;
a header comprising an exterior surface that encloses an interior region; and
an integrated electrode/antenna assembly at least partially disposed within the interior region of the header.

19. The IMD of claim 18, wherein the integrated electrode/antenna assembly comprises an antenna component and an electrode component.

20. The IMD of claim 19, wherein the antenna component comprises:
a driven element electrically coupled to the circuitry; and
a reflector comprising a ground-plane disposed on at least a portion of an internal surface of the header, wherein the electrode component functions as a director.
